(19) United States
(12) Patent Application Publication
(10) Pub. No.: US 2022/0215950 A1
(43) Pub. Date: Jul. 7, 2022

(54) VACCINATION AND TESTING VALIDATION AND VERIFICATION

(71) Applicant: Azova, Inc., Alpine, UT (US)

(72) Inventor: Cheryl Lee Eberting, Alpine, UT (US)

(21) Appl. No.: 17/551,134

(22) Filed: Dec. 14, 2021

Related U.S. Application Data
(60) Provisional application No. 63/125,227, filed on Dec. 14, 2020.
(60) Provisional application No. 63/125,366, filed on Dec. 14, 2020.
(60) Provisional application No. 63/179,181, filed on Apr. 23, 2021.

(51) Int. Cl. G16H 40/67; G06K 19/06

(52) U.S. Cl. CPC G16H 40/67; G06K 19/06037

(57) ABSTRACT

The present disclosure provides systems, methods, and workflows related to the validation and sharing of health credentials, including vaccination statuses. A single QR code is generated to provide trusted and third-party verification of vaccination and test status of a user, even when the vaccination and testing were performed by different entities.

### RELATED APPLICATIONS

This application claims priority to and benefit under 35 U.S.C. § 119 to U.S. Provisional Patent Application No. 63/125,227 filed on Dec. 14, 2020, titled “Systems, Methods, and Workflows for Self-Administered Testing;” U.S. Provisional Patent Application No. 63/125,366 filed on Dec. 14, 2020, titled “Third-Party Redemption of Self-Administered Test Kits Purchased Through a Marketplace;” and U.S. Provisional Patent Application No. 63/179,181 filed on Apr. 23, 2021, titled “Vaccination and Testing Validation and Verification,” each of which is hereby incorporated by reference in its entirety.

### TECHNICAL FIELD

This disclosure relates to vaccine management, validation, and verification involving multiple entities and interested parties. This disclosure also relates to maintaining personal information confidential and private.

### BRIEF DESCRIPTION OF THE DRAWINGS

Non-limiting and non-exhaustive embodiments of the disclosure are described herein, including various embodiments of the disclosure with reference to the figures listed below.

FIGS. 1A-1L illustrate a first workflow that includes the test kit being shipped after the lab order is created, according to one embodiment.

FIGS. 2A-2M illustrate another workflow that includes the test kit being shipped and the patient getting the lab order before taking the test, according to one embodiment.

FIGS. 3A-3B illustrate an example graphical user interface (GUI) and a computer-implemented process for sharing a validated covid vaccination record, according to various embodiments.

FIGS. 4A-4B illustrate example GUIs for initiating a vaccine record validation and uploading a photo identification, according to various embodiments.

FIGS. 5A-5D illustrate an example computer-implemented process for sharing a validated covid vaccination record, according to various embodiments.

FIGS. 6A-6F illustrated example GUIs for a user to input vaccine information that can be used by the system for validation, according to one embodiment.

FIG. 7 illustrates an example GUI for user-management and creation of vaccine records that can be validated and shared, according to one embodiment.

FIGS. 8 illustrates example GUIs of a system for sharing a validated vaccination record, test results, or other status, according to various embodiments.

FIG. 9 illustrates an example GUI for a user to share vaccination credentials (or any other health credential) as a photo, via an app, via email, as a printed PDF, or as QR code, according to one embodiment.

FIG. 10 illustrates an example of a computer system 1000, according to one embodiment.

### DETAILED DESCRIPTION

Various embodiments of this disclosure can be incorporated into, used by, and/or used in conjunction with one or more of the embodiments or combination of embodiments described in U.S. patent application Ser. No. 15/597,102 titled “Telemedicine Platform with Integrated e-Commerce and Third Party Interfaces,” filed on May 16, 2017 (the “'102 Application” and/or PCT Application No. PCT/US2016/020964 titled “Telemedicine Platform and Associated Services with Third-Party Interfaces,” filed on Mar. 4, 2016, both of which are hereby incorporated by reference in their entireties. Similarly, the infrastructure and underlying principles described the incorporated applications can be used to implement and/or augment the systems and methods described herein.

Some of the infrastructure that can be used with embodiments disclosed herein is already available, such as: general-purpose computers, computer programming tools and techniques, digital storage media, and communications networks. A computer may include a processor, such as a microprocessor, microcontroller, logic circuitry, or the like. The processor may include a special-purpose processing device, such as an ASIC, a PAL, a PLA, a PLD, a CPLD, a Field Programmable Gate Array (FPGA), or other customized or programmable device. The computer may also include a computer-readable storage device, such as non-volatile memory, static RAM, dynamic RAM, ROM, CD-ROM, disk, tape, magnetic, optical, flash memory, or other computer-readable storage medium.

Suitable networks for configuration and/or use, as described herein, include any of a wide variety of network infrastructures. Specifically, a network may incorporate landlines, wireless communication, optical connections, various modulators, demodulators, small form-factor pluggable (SFP) transceivers, routers, hubs, switches, and/or other networking equipment.

The network may include communications or networking software, such as software available from Novell, Microsoft, Artisoft, and other vendors, and may operate using TCP/IP, SPX, IPX, SONET, and other protocols over twisted pair, coaxial, or optical fiber cables; telephone lines; satellites; microwave relays; modulated AC power lines; physical media transfer; wireless radio links; and/or other data transmission “wires.” The network may encompass smaller networks and/or be connectable to other networks through a gateway or similar mechanism.

Aspects of certain embodiments described herein may be implemented as software modules or components. As used herein, a software module or component may include any type of computer instruction or computer-executable code located within or on a computer-readable storage medium, such as a non-transitory computer-readable medium. A software module may, for instance, comprise one or more physical or logical blocks of computer instructions, which may be organized as a routine, program, object, component, data structure, etc., that perform one or more tasks or implement particular data types, algorithms, and/or methods.

A particular software module may comprise disparate instructions stored in different locations of a computer-readable storage medium, which together implement the described functionality of the module. Indeed, a module may comprise a single instruction or many instructions, and may be distributed over several different code segments, among different programs, and across several computer-readable storage media. Some embodiments may be practiced in a distributed computing environment where tasks are performed by a remote processing device linked through a communications network. In a distributed computing environment, software modules may be located in local and/or remote computer-readable storage media. In addition, data being tied or rendered together in a database record may be resident in the same computer-readable storage medium, or across several computer-readable storage media, and may be linked together in fields of a record in a database across a network.

The various functional components of the described systems and methods may be modeled as a functional block diagram that includes one or more remote terminals, networks, servers, data exchanges, and software/hardware/firmware modules configured to implement the various functions, features, methods, and concepts described herein. In many instances, each application, embodiment, variation, option, service, and/or other component of the systems and methods described herein may be implemented as a module of a larger system. Each module may be implemented as hardware, software, and/or firmware, as would be understood by one of skill in the art for the particular functionality, and may be part of a larger physical system that may include computer-readable instructions, processors, servers, endpoint computers, and/or the like.

The embodiments of the disclosure can be understood by reference to the drawings, wherein like parts are designated by like numerals throughout. The components of the disclosed embodiments, as generally described and illustrated in the figures herein, could be arranged and designed in a wide variety of different configurations. Further, those of skill in the art will recognize that one or more of the specific details may be omitted, or other methods, components, or materials may be used. In some cases, operations are not shown or described in detail. Thus, the following detailed description of the embodiments of the systems and methods of the disclosure is not intended to limit the scope of the disclosure, as claimed, but is merely representative of possible embodiments.

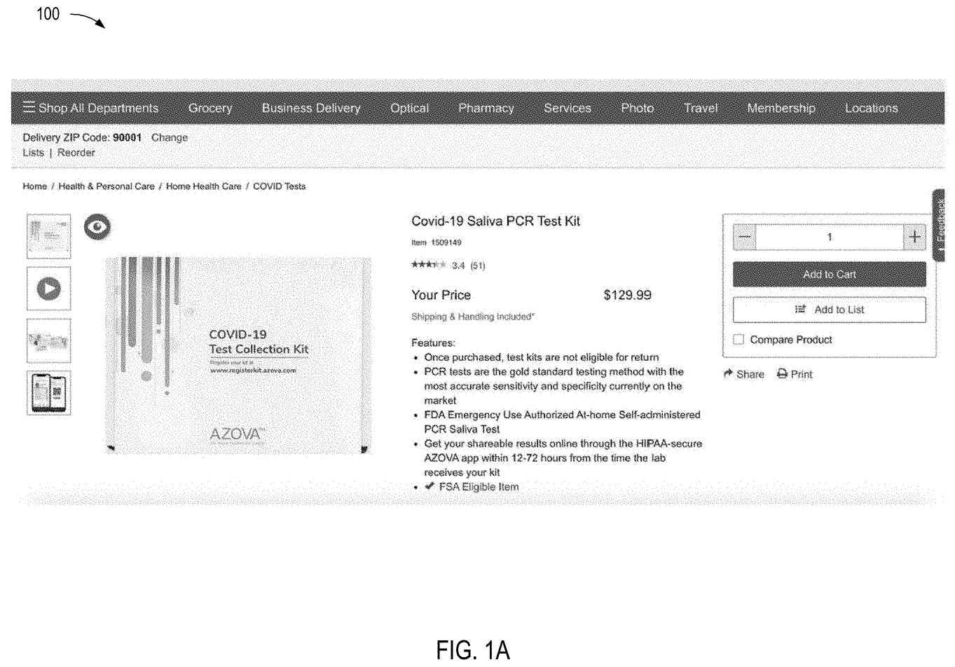

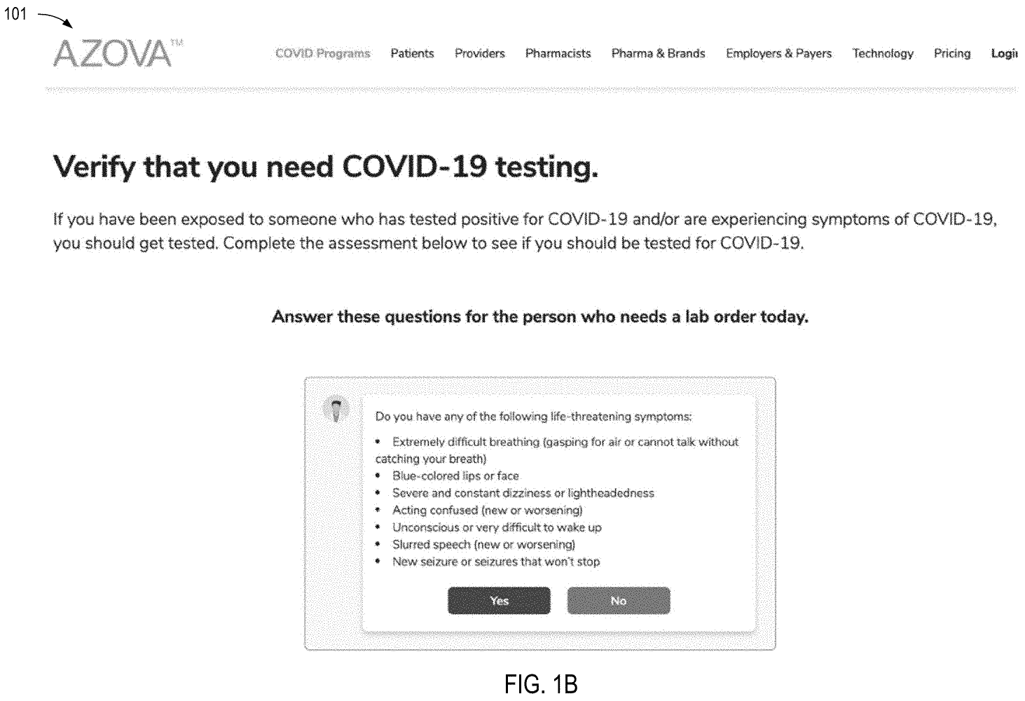

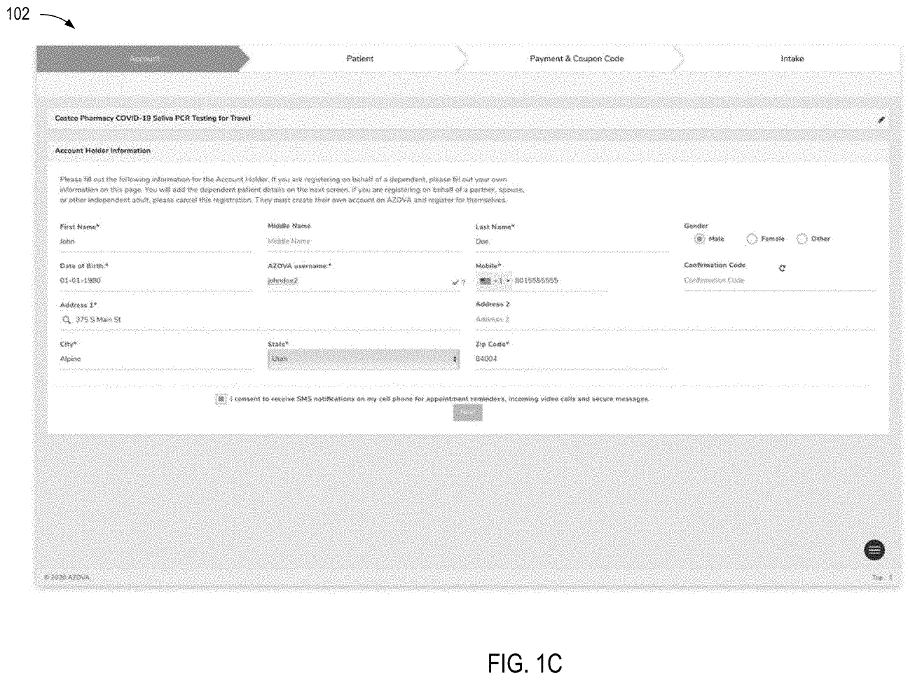

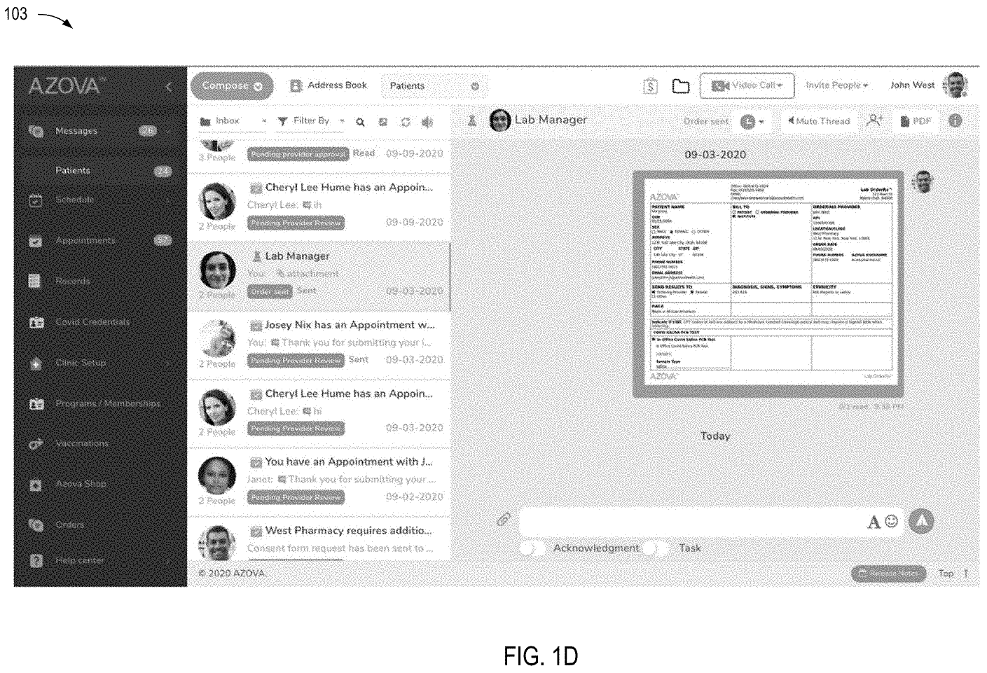

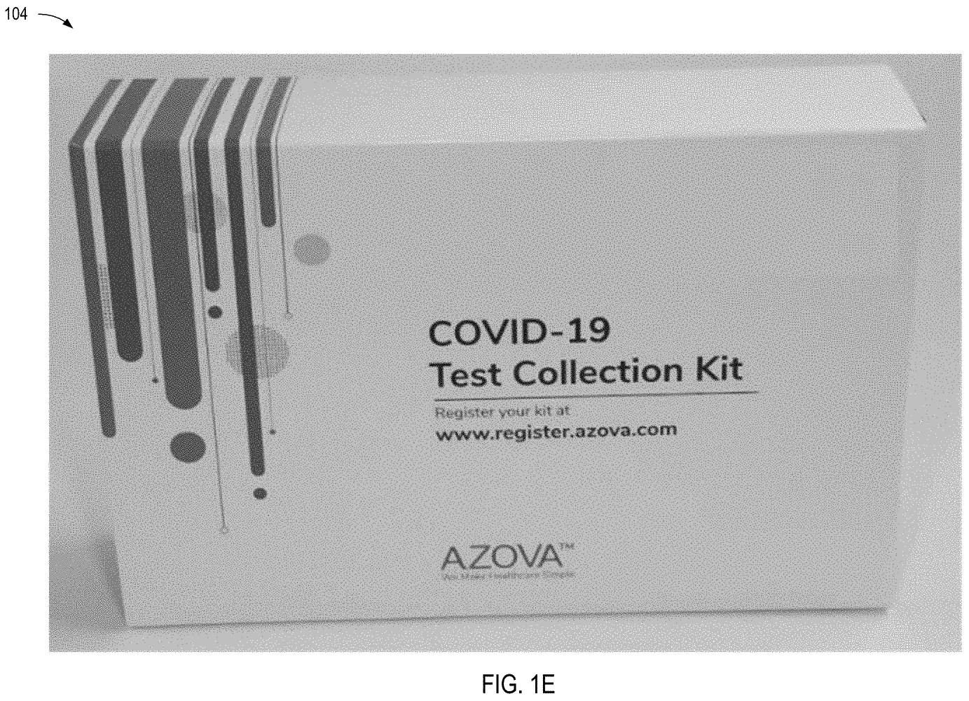

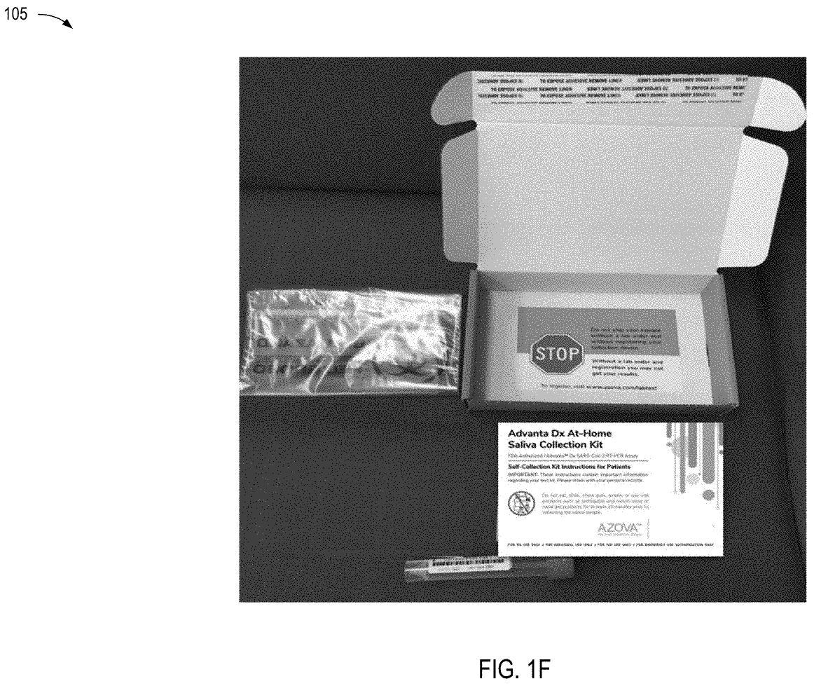

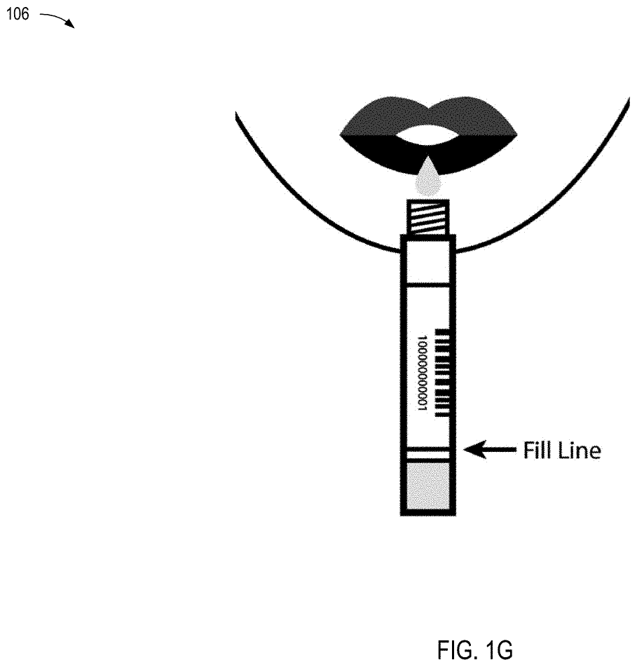

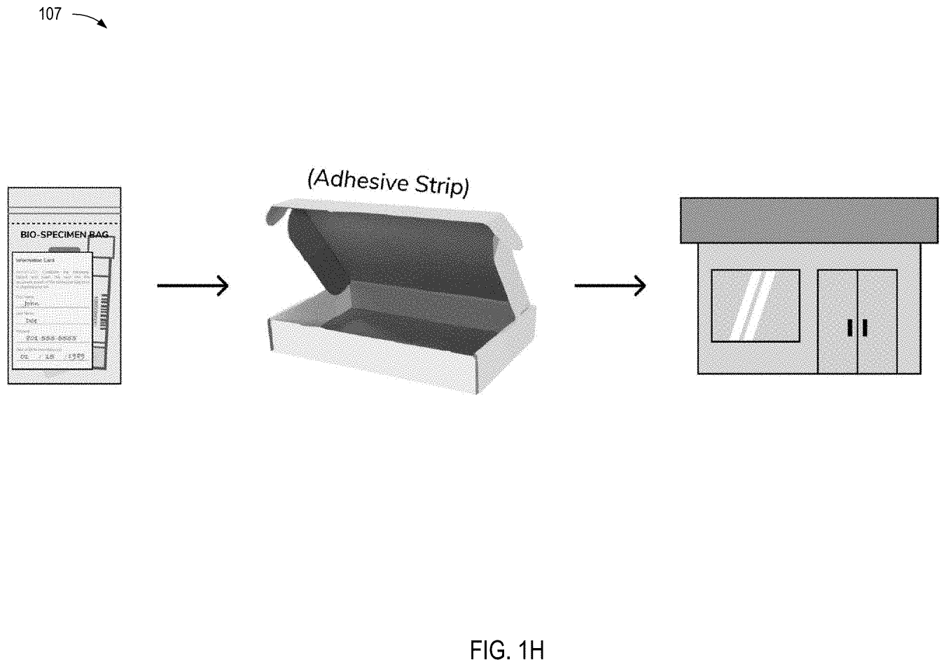

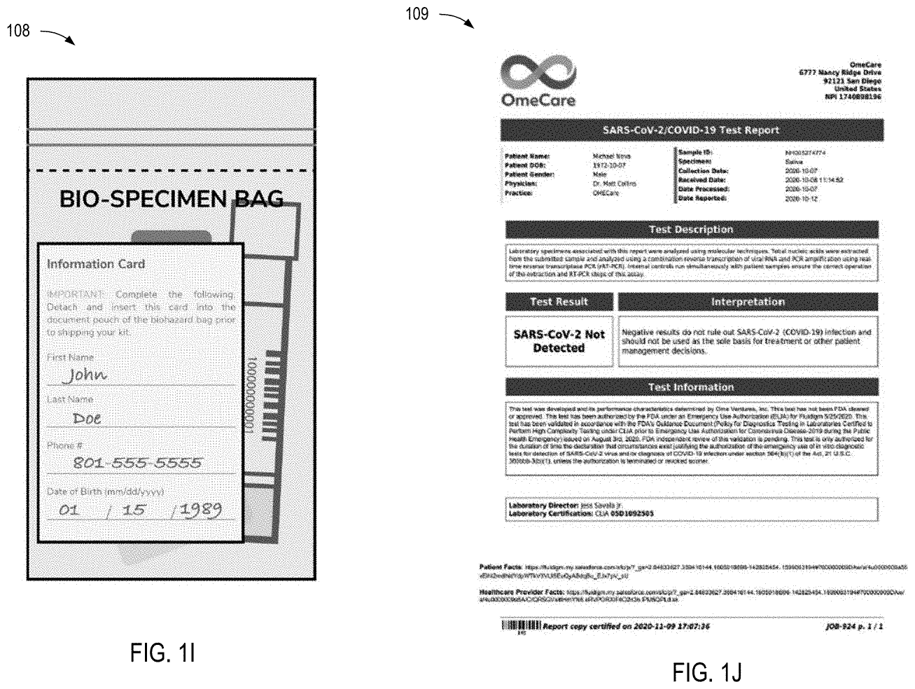

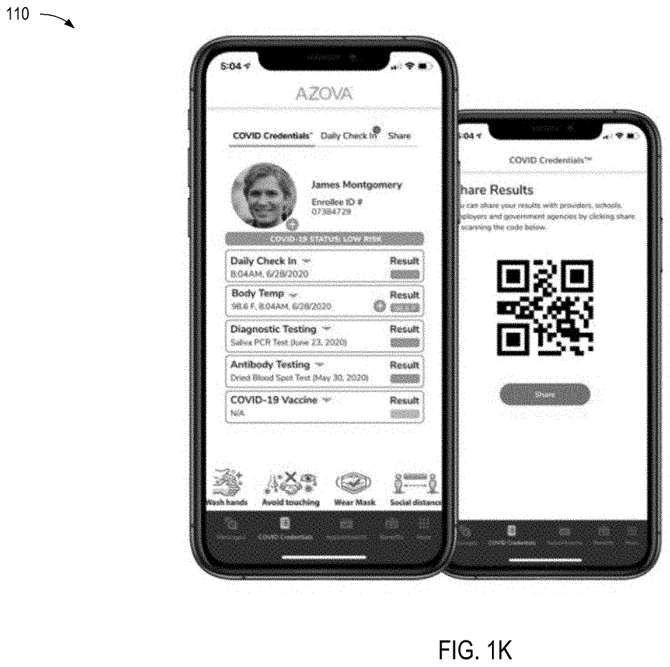

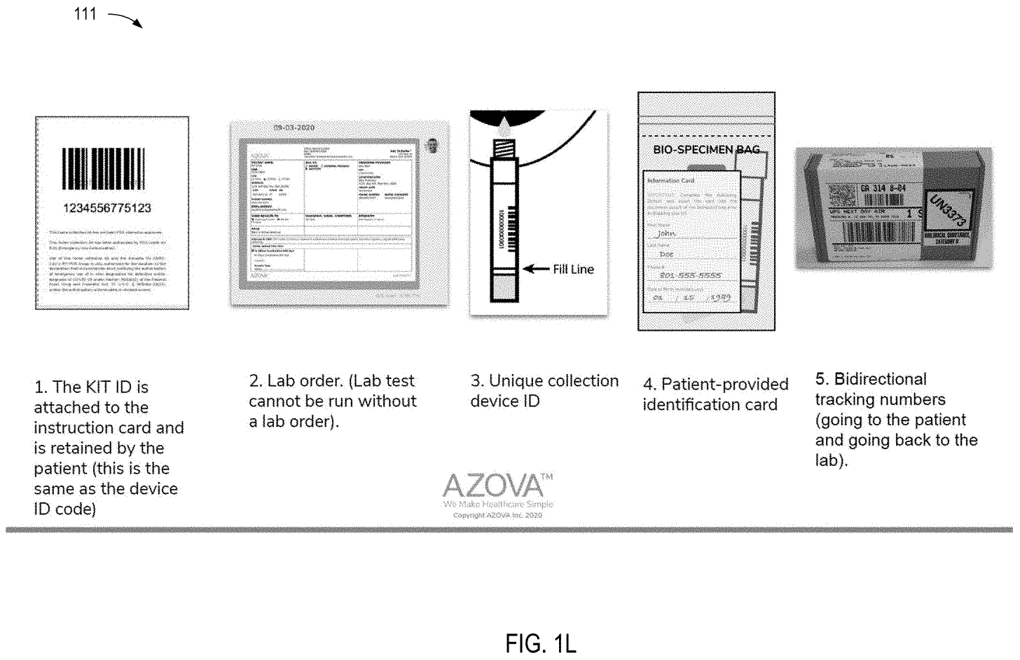

FIG. 1A illustrates a first step 100 in which a patient purchases a test from an online retailer or a branded AZOVA microsite. The kit is not shipped until the patient completes a health assessment and health intake form and gets a lab order. (e.g., for COVID-19 or any other test).

FIG. 1B illustrates a second step 101 in which, before the kit is shipped, the patient must complete a health assessment and, if testing is deemed appropriate, a health intake form to get a prescription lab order.

FIG. 1C illustrates a third step 102 in which a user creates a secure account on AZOVA and completes a health intake form and provides demographic information that is used for state reporting and gets a telemedicine lab order.

FIG. 1D illustrates a fourth step 103 in which the lab order is transmitted to the lab from the ordering provider. The patient is notified that the order has been placed.

FIG. 1E illustrates a fifth step 104 in which the COVID-19 test kit is shipped to the patient, according to one embodiment.

FIG. 1F illustrates the contents of an example kit 105 that includes, one kit box with attached return, shipping label; instructions for use with embedded lab order coupon code and attached information sheet; sterile collection device with attached unique UPC ID number (Kit ID); biohazard bag with absorbent sheet; and a sticker to remind patient to register their collection device ID before returning it.

FIG. 1G illustrates a sixth step 106 in which a patient follows instructions to provide a saliva sample and register the test kit ID.

FIG. 1H illustrates a seventh step 107 in which the patient puts biohazard bag in return shipping box and drops off kit at the appropriate shipping carrier. Return shipping label is attached to the box making a simple patient experience.

FIG. 1I illustrates an eight step 108 in which the lab receives the kit on the next business day (including Saturdays) and processes the sample. The patient is notified when the sample is accessioned by the lab.

FIG. 1J illustrates a ninth step 109 in which the patient quickly receives results via a secure email and SMS link on AZOVA.com. The patient can log in to view and share their results. AZOVA transmits the patient's demographic information to the laboratory and the laboratory combines this with the test results and reports them to the respective reporting agency. The Fact Sheet for Healthcare Providers and the Fact Sheet for Patients is included on the lab report.

FIG. 1K illustrates a tenth step 110 in which AZOVA (e.g., via a computer system) dynamically creates a COVID Credential for the patient. This is a laboratory agnostic longitudinal record of COVID diagnostic testing, antibody testing, and COVID immunizations. Lab results that are received through AZOVA are immutable by the patient and are electronically validated. The patient can share their COVID Credentials with anyone they like.

FIG. 1L illustrates a chain of custody and tracking system 111 that provides multipole layers of sample tracking, according to a first set of embodiments.

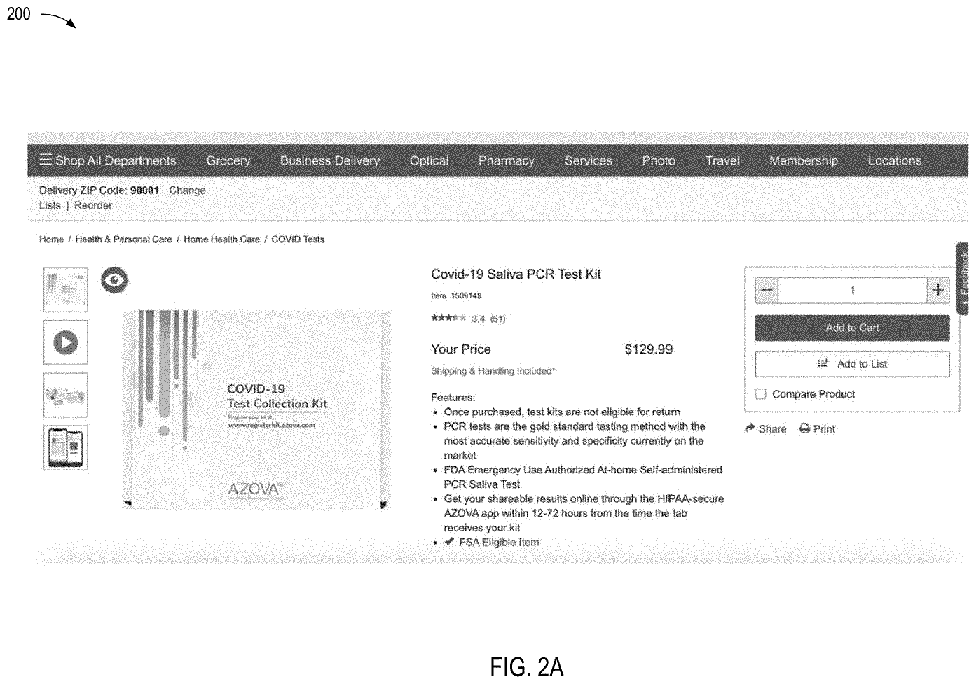

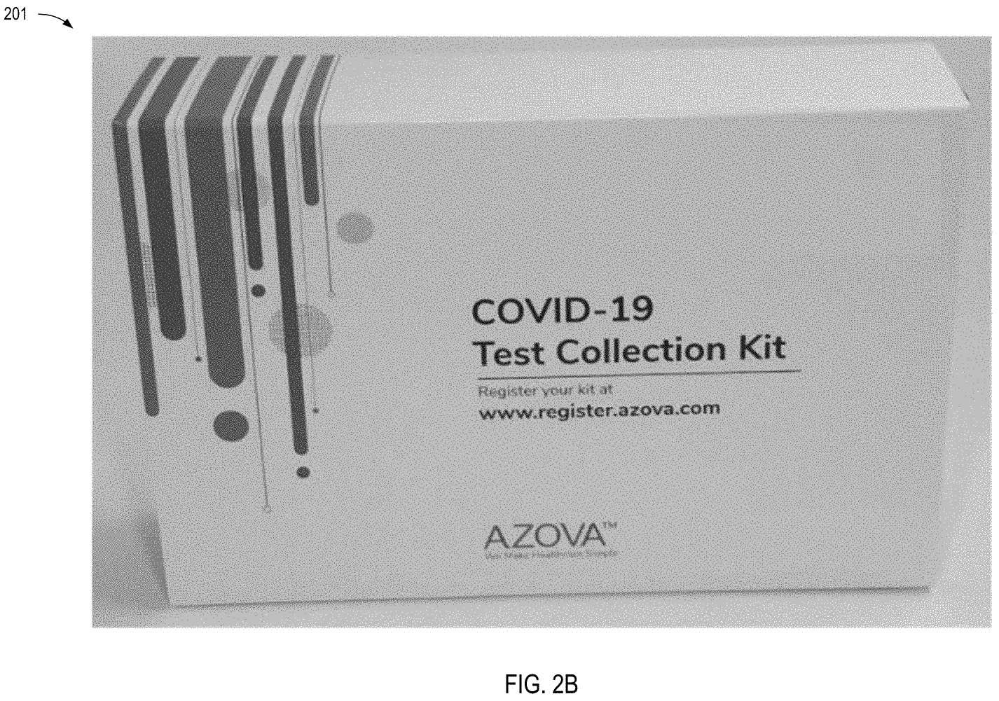

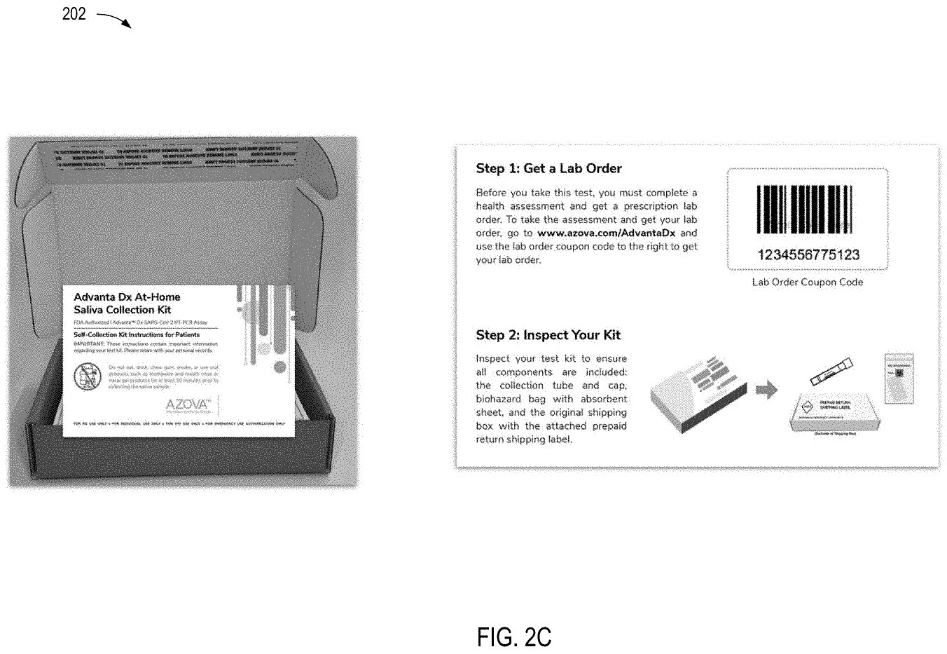

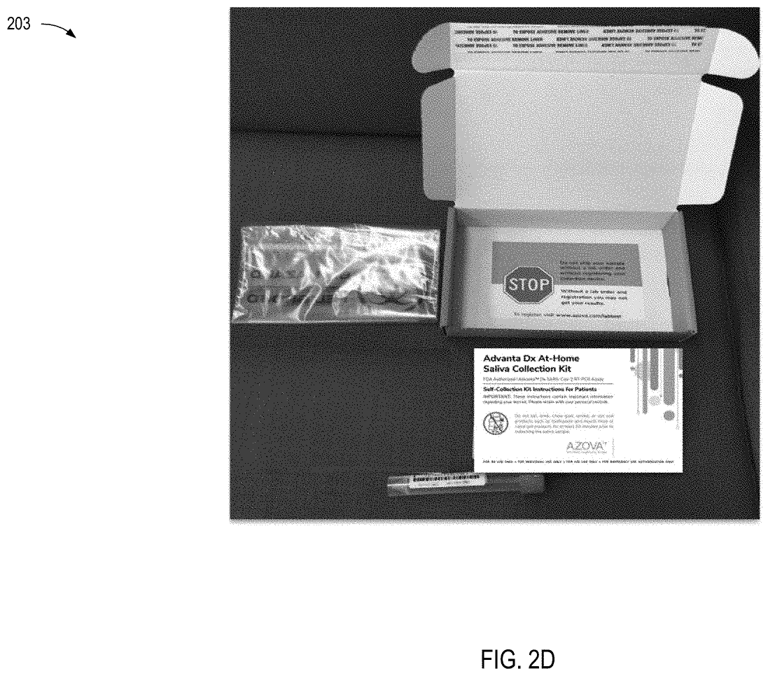

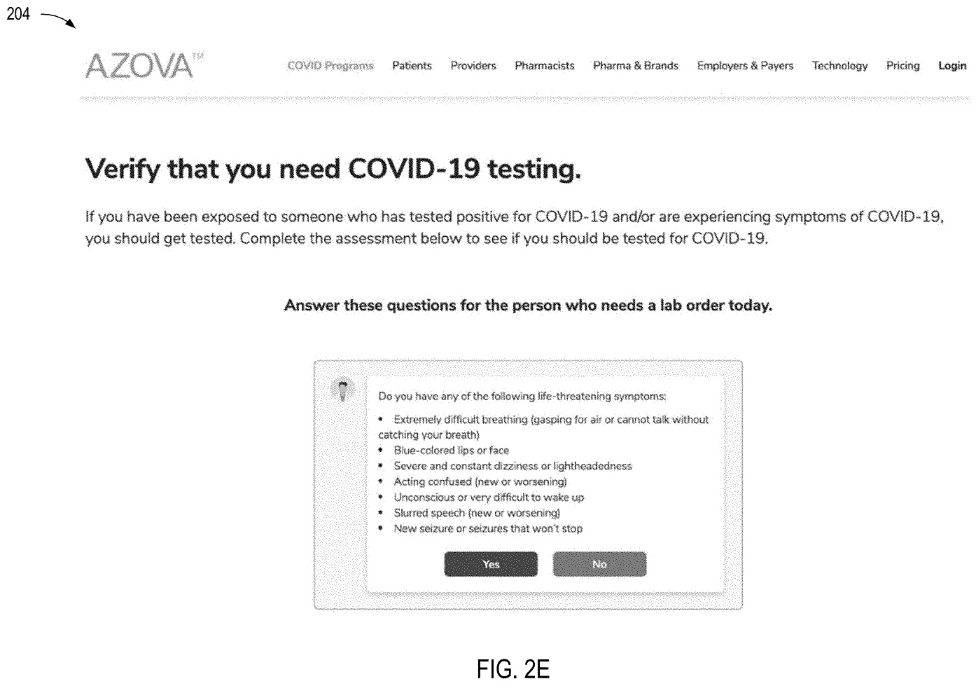

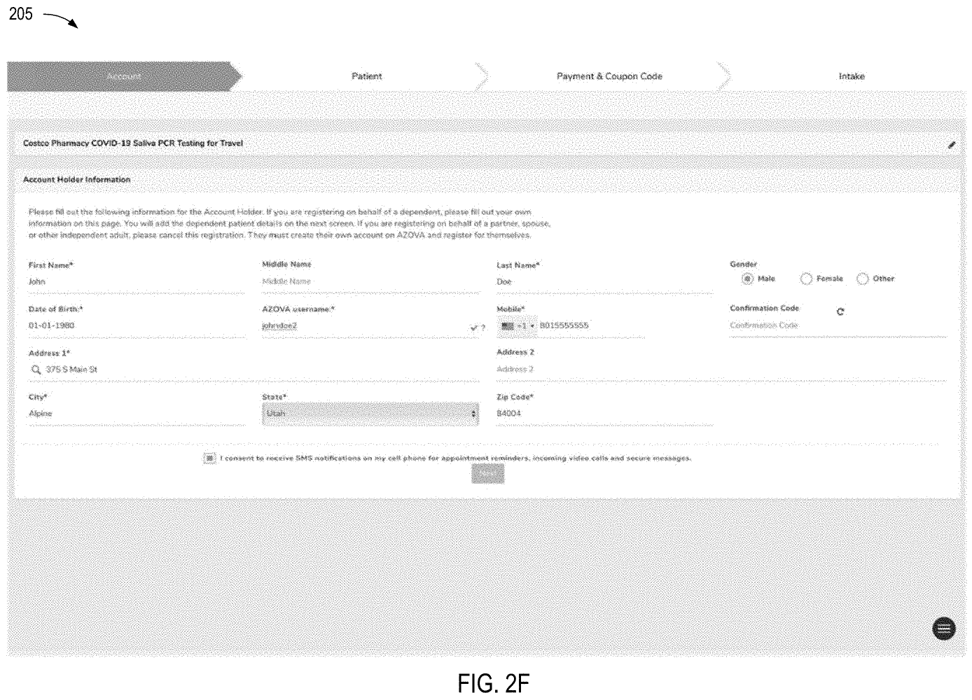

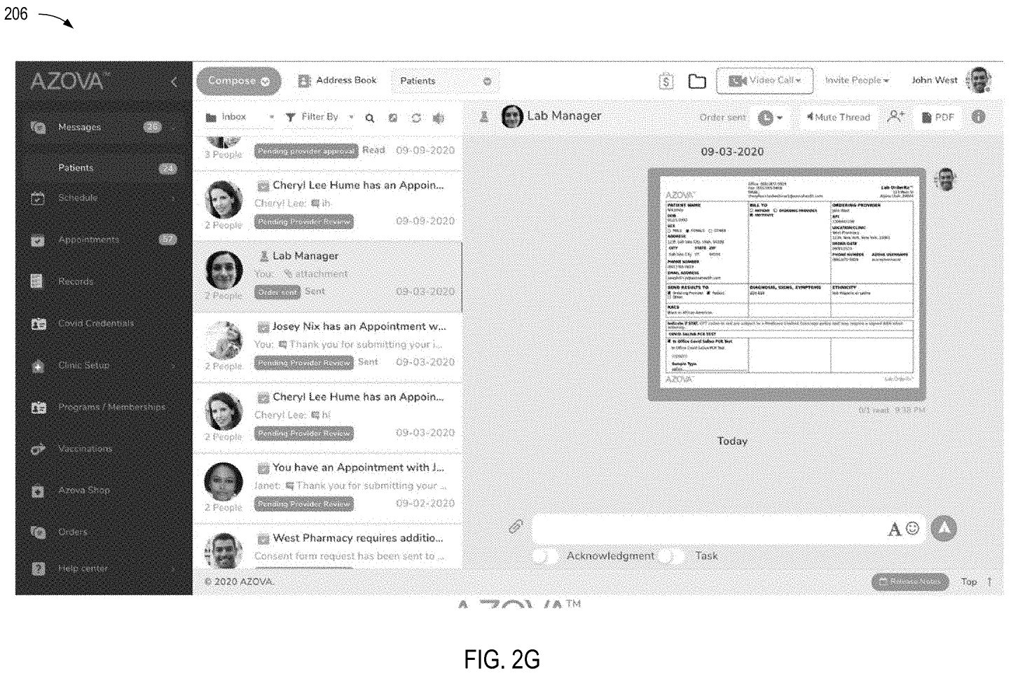

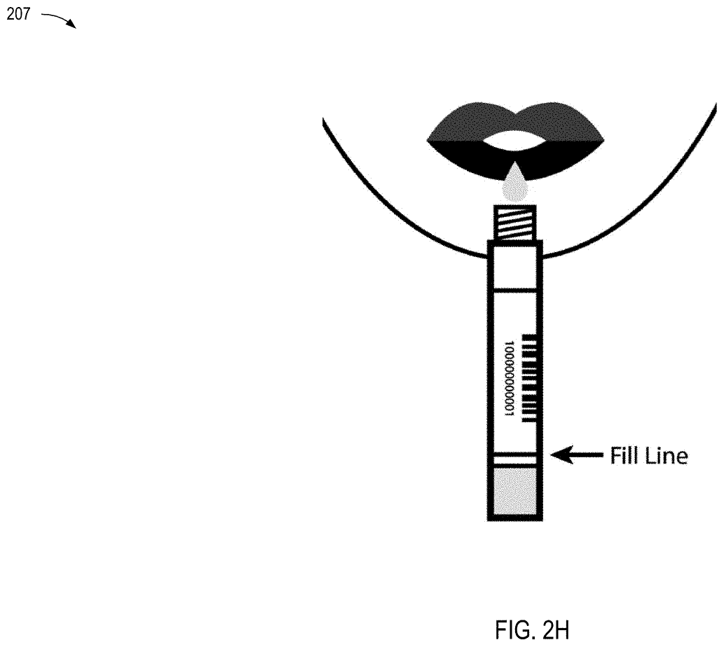

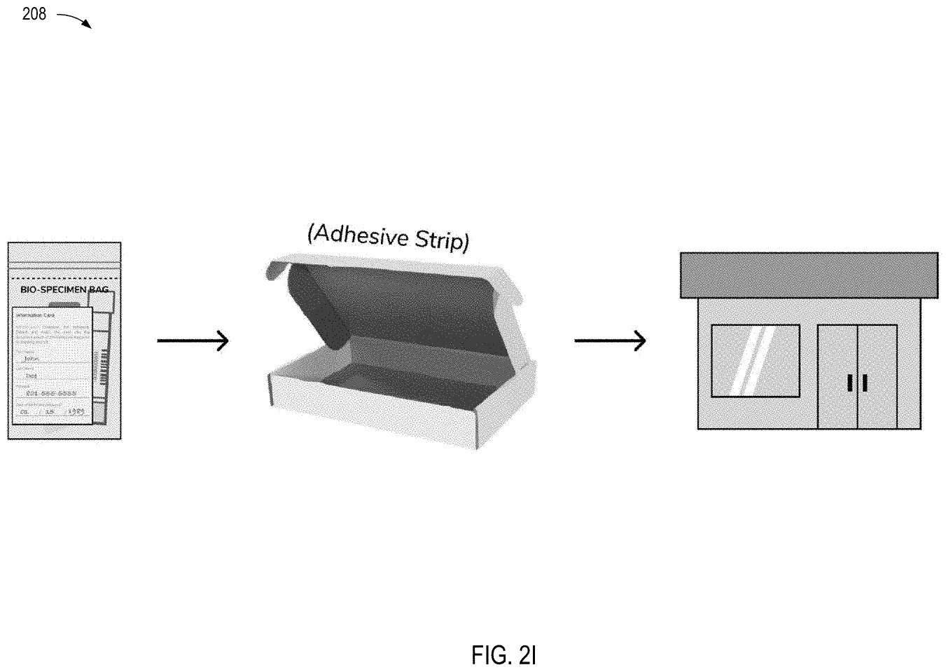

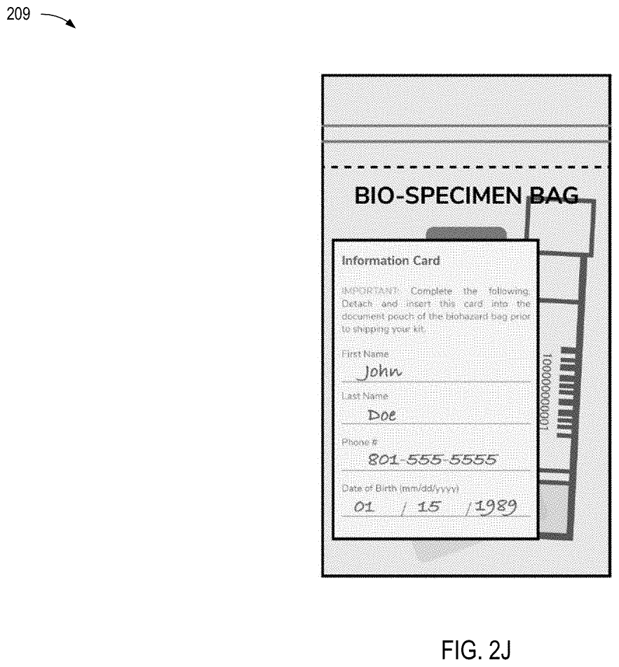

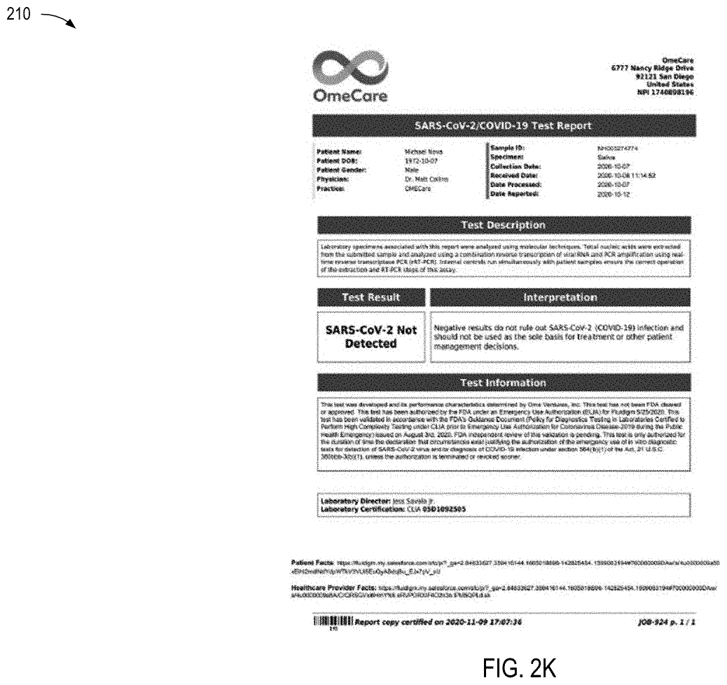

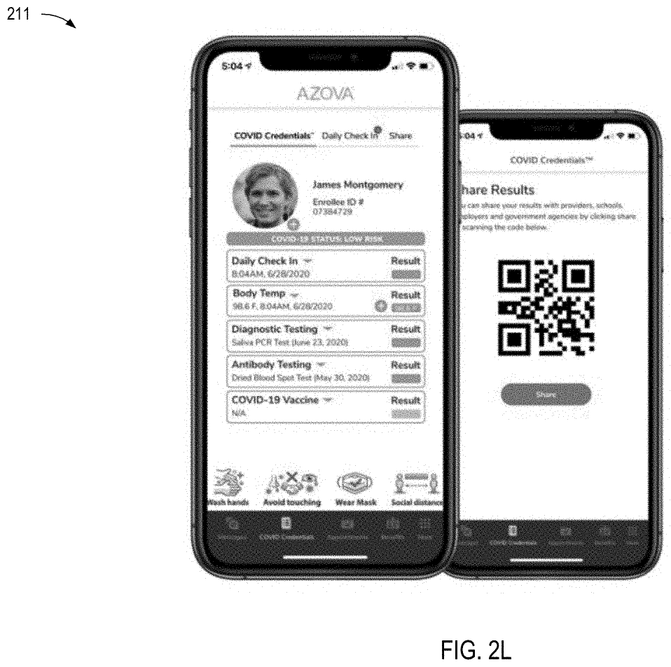

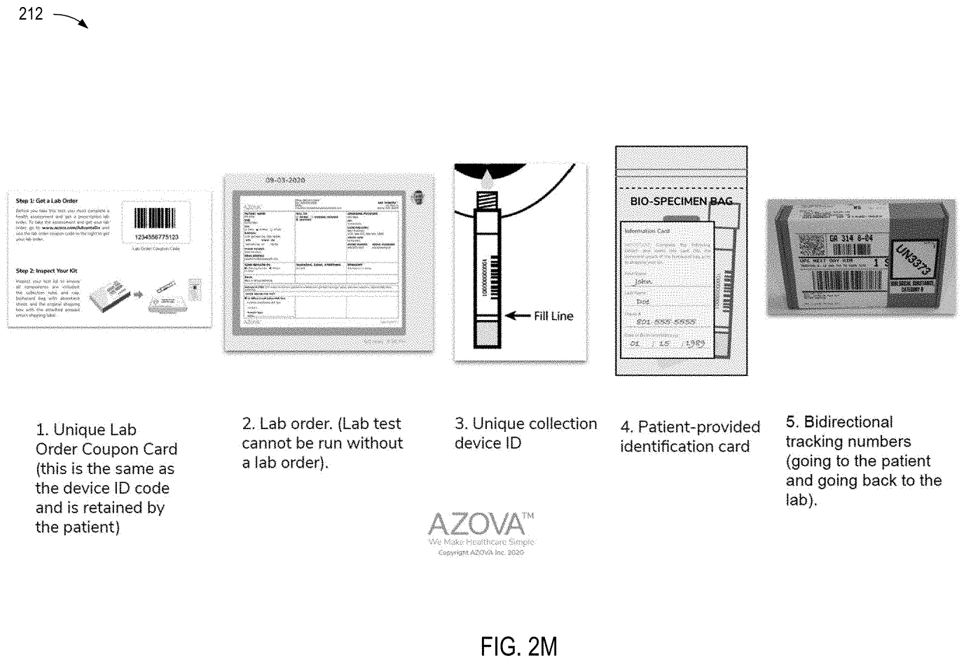

FIG. 2A illustrates an example first step 200 of another approach in which a patient purchases a test from an online retailer or walks into a retail pharmacy or other retail location and purchases a test kit.

FIG. 2B illustrates a next step 201 in which the COVID-19 test kit is shipped to the patient, according to one embodiment.

FIG. 2C illustrates a step 202 in which, included inside the test kit is the instructions (e.g., AdvantaDx instructions for use card) with embedded lab order coupon code. Before the patient can take the test, the patient must complete a health assessment and, if testing is appropriate, a health intake form to get a lab order prescription.

FIG. 2D illustrates the contents of an example kit 203 that includes, one kit box with attached return, shipping label; instructions for use with embedded lab order coupon code and attached information sheet; sterile collection device with attached unique UPC ID number (Kit ID); biohazard bag with absorbent sheet; and a sticker to remind patient to register their collection device ID before returning it.

FIG. 2E illustrates a step 204 in which the patient goes to registration URL and completes a CDC-guided health assessment. If testing is appropriate, patient can continue to complete a health intake form and get a lab order.

FIG. 2F illustrates a step 205 in which the user creates a secure account on AZOVA and enters the lab order coupon code and completes a health intake form and provides demographic information that is used for state reporting and gets a telemedicine lab order.

FIG. 2G illustrates a step 206 in which the lab order is transmitted to the lab.

FIG. 2H illustrates the patient following instructions 207 to provide a saliva sample and register the test kit ID.

FIG. 2I illustrates a step 208 in which the patient puts biohazard bag in return shipping box and drops off kit at the appropriate shipping carrier. Return shipping label is attached to the box making a simple patient experience.

FIG. 2J illustrates a step 209 in which the lab receives the kit on the next business day (including Saturdays) and processes the sample. The patient is notified when the sample is accessioned by the lab.

FIG. 2K illustrates a step 210 in which the patient quickly receives results via a secure email and SMS link on AZOVA.com. The patient can log in to view and share their results. AZOVA transmits the patient's demographic information to the laboratory and the laboratory combines this with the test results and reports them to the respective reporting agency. The Fact Sheet for Healthcare Providers and the Fact Sheet for Patients is included on the lab report.

FIG. 2L illustrates a step 211 in which AZOVA (e.g., via a computer system) dynamically creates a COVID Credential for the patient. This is a laboratory agnostic longitudinal record of COVID diagnostic testing, antibody testing, and COVID immunizations. Lab results that are received through AZOVA are immutable by the patient and are electronically validated. The patient can share their COVID Credentials with anyone they like.

FIG. 2M illustrates another chain of custody and tracking system 212that provides multipole layers of sample tracking, according to another set of embodiments.

Various embodiments of the presently described systems and methods facilitate electronic vaccination record validation, verification, and sharing. For example, a user may create an account and consent to share a vaccination record with one or more individuals, governments, companies, and/or other entities. The system may request that the user provide details regarding their vaccination to facilitate automatic or manual validation of the vaccination by the system, the administrator of the vaccine, and/or a trusted repository or database of vaccination records.

The system may charge a fee to validate a user's vaccination. In some instances, a given vaccination may have multiple doses or boosters and each dose and or booster may be validated separately or as a group or set of vaccines. The system may receive the vaccination details provided by the user and use them for validation purposes. In some embodiments, the system may request (e.g., by phone, email, digital request, website query, database query, via an API call, or the like) that the administrator of the vaccine (as identified by the user) validate the vaccination details provided by user and/or provide additional details regarding the vaccination. In some embodiments, other vaccination records maintained or known about by the vaccine administrator or database may be provided as well.

The system marks the user's vaccination record as “validated” in response to the vaccine administrator's validation and accompanying vaccine information. The user may manually show (e.g., via a display on a screen or a printed document) the validated vaccination to an interested party. In some embodiments, the validated vaccine information may only include limited information sufficient for the interested party (that trusts the system) to verify that the user has a validated vaccination.

For example, a user may obtain two doses of a Covid-19 vaccine. The user may upload details of each dose of the Covid-19 vaccine to the system. The system may contact the administrator of each dose of the Covid-19 vaccine and/or query a database of records to validate the user's Covid-19 vaccination status. The user may then elect to share the validated vaccination status with one or more individuals, governments, organizations, employers, countries, travel providers, companies, medical professionals, schools, entertainment venues, etc.

According to various embodiments, the user may add dependents (e.g., children) and validate and share their vaccination records as well. According to some embodiments, a user may present a QR code to be scanned by an individual, a government, an organization, an employer, a country, a travel provider, a company, a medical professional, a school, a facility, an entertainment venue, or other scanning entity. The scanning entity may request verification that the user has obtained a particular vaccine, specific test result, temperature reading, self-certification of no symptoms, or the like.

In some instances, the scanning entity may be satisfied with a user-certified statement made that same day (or during another time period) that they have no symptoms, have a normal temperature reading, have been in quarantine, are wearing a mask or other PPE, have followed guidelines or laws, or the like. In other instances, the scanning entity may wish to verify that the user has self-certified as having received a particular test result (e.g., a negative test result) or a specific vaccine or set of vaccines. In still other instances, the scanning entity may wish to verify that the system has validated (e.g., trusted third-party validation) vaccination records, test results, temperature readings, or other “health credential.”

The user submits details of a health credential. The system validates the health credential. The system allows the user to share the health credential or share a verification that thy system has validated the health credential. The user and/or the system may control how much personal information is shared in conjunction with the health credential. For example, system may provide a confirmation of a validated health credential and nothing more. In other embodiments, the system may provide a confirmation of a validated health credential and a photo for real-time verification that that validated health credential belongs to the person presented the validated health credential. In other embodiments, the system may provide personal information and/or additional information regarding the health credential as requested or demanded by the receiving entity and/or as allowed or authorized by the user. The system aggregates all the sources of data, including the immunization registry, the health department records, pharmacy records, doctor records, hospital records, and the like. The aggregated data is stored in a user-controlled account. The credential status can be shared with or without personal information of the user. The user can control the data, how it is shared, and when it is shared.

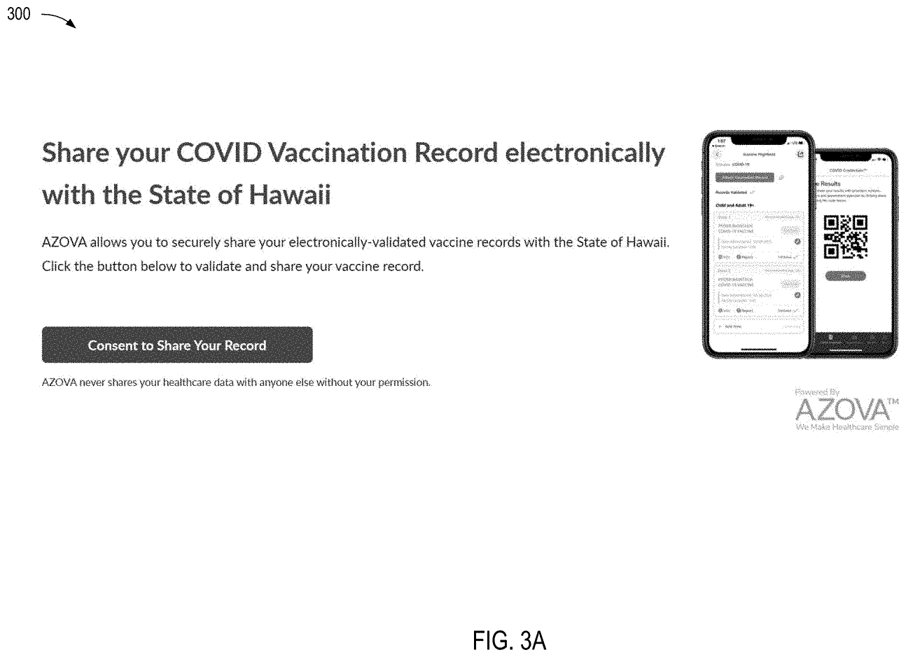

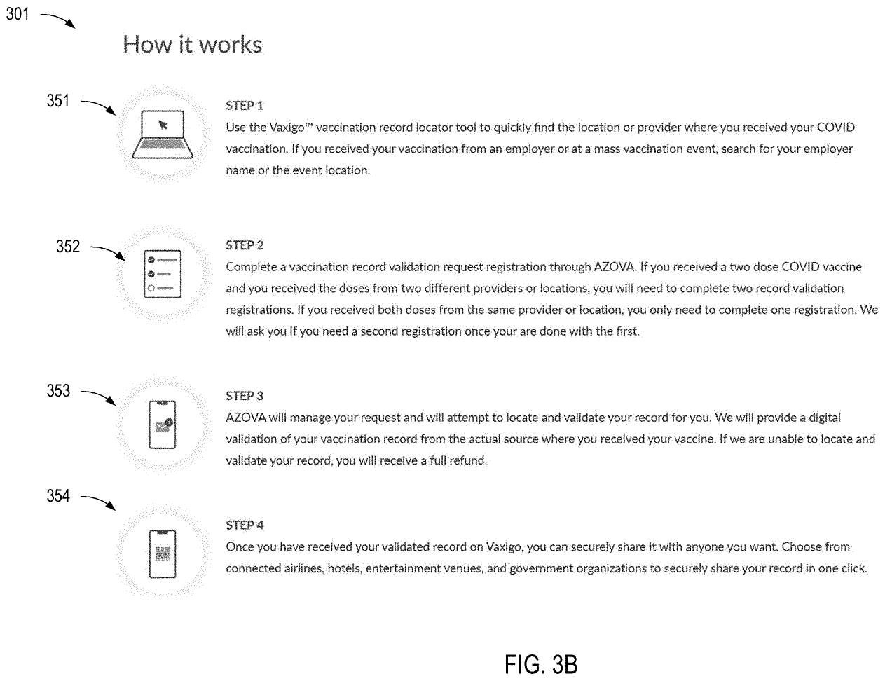

FIG. 3A illustrate an example graphical user interface (GUI) 300 for sharing a validated covid vaccination record, according to various embodiments. As illustrated, a user may consent to share a health credential with a government or associated entity. For example, the user may share a validated vaccination record of a Covid-19 vaccine with a state or country for purposes of work or travel.

FIG. 3B illustrates a computer-implemented process 301 for sharing a validated covid vaccination record, according to various embodiments. As illustrated, a user may use a Vaxigo vaccination record locator tool (e.g., a module of a non-transitory computer-readable medium portion of a system) to identify the location or provider where a vaccine (or other health credential) was received, at 351.

The user may then request that the system validate the vaccine or other health credential, at 352. The system manages the request, locates your records, contacts necessary personal for validation, queries available databases, utilizes APIs when available, and/or otherwise confirms or validates the existence and other details of the particular health credential, at 353. The user can then share the validated record using the Vaxigo tool with any individual or entity, at 354. Sharing the validated record may include only the transmission by the system to the entity that the health credential exists and has been validated by the system. Accordingly, the specific personal information of the user and/or the personal and specific details of the health record may not necessarily be included in the information shared.

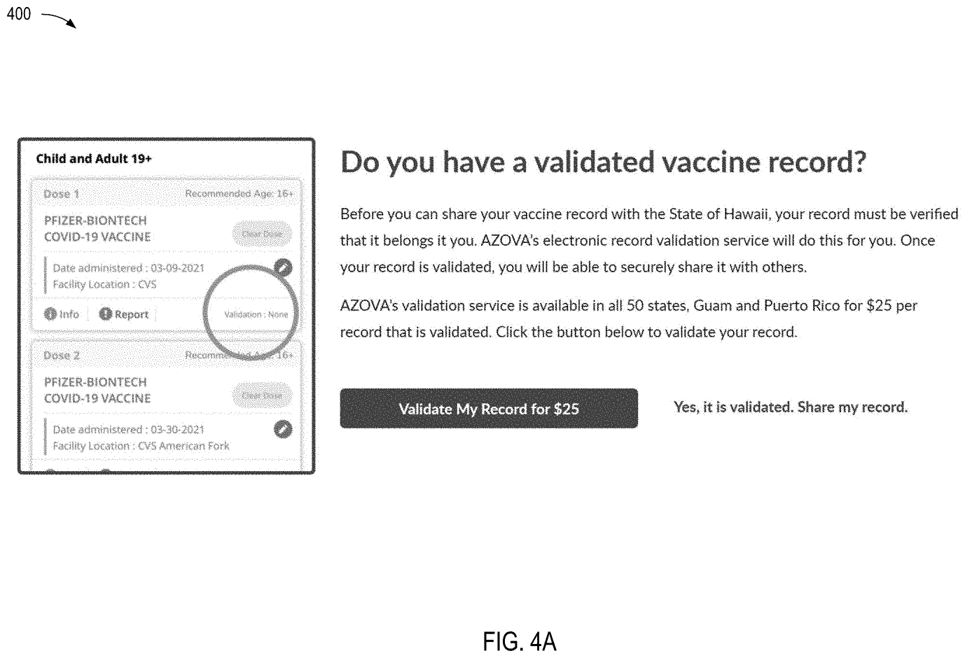

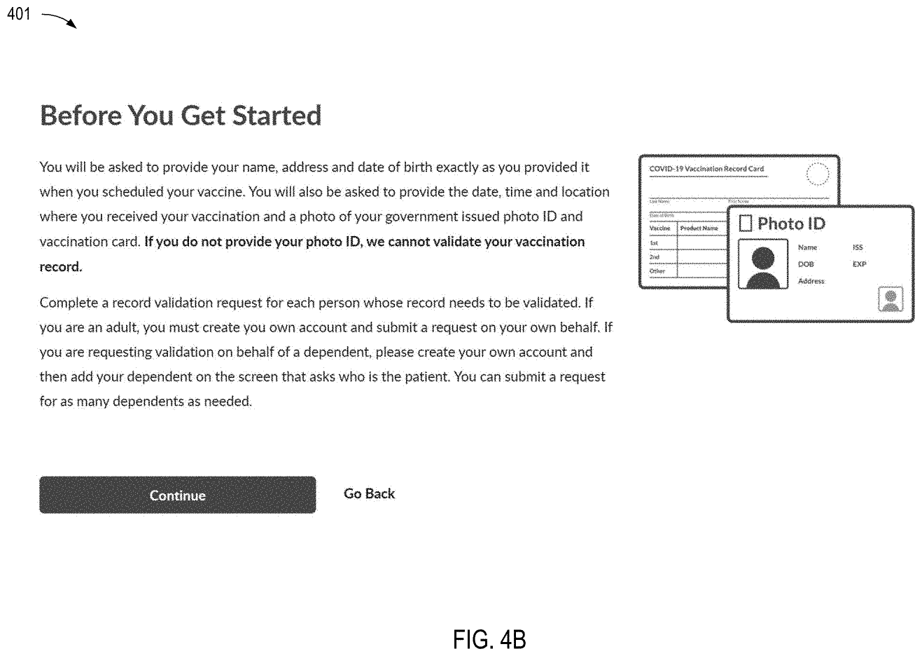

FIG. 4A illustrates an example GUI 400 for initiating a vaccine record validation by paying a fee.

FIG. 4B illustrates an example GUI 401for the user to upload a photo identification for the user and/or dependents of the user, according to various embodiments.

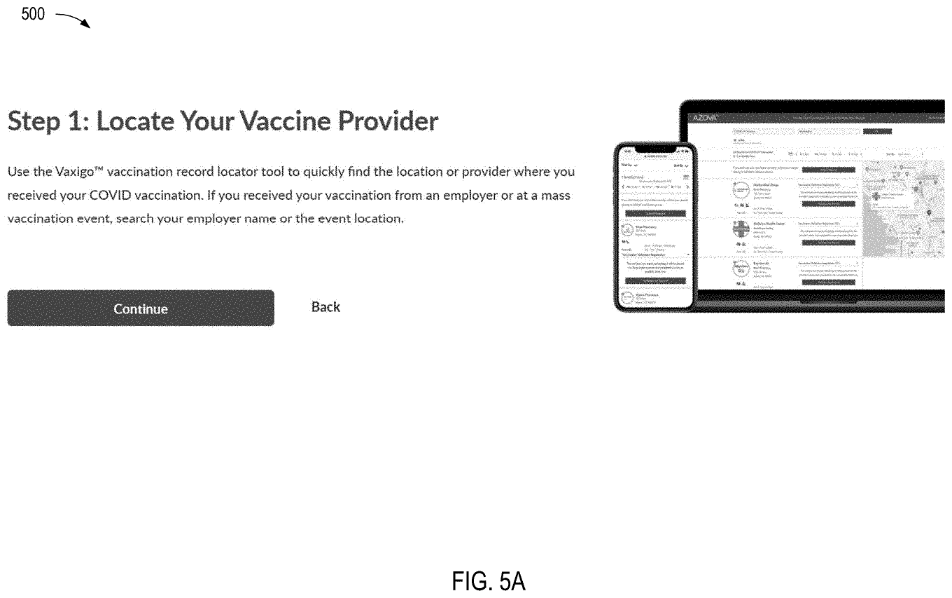

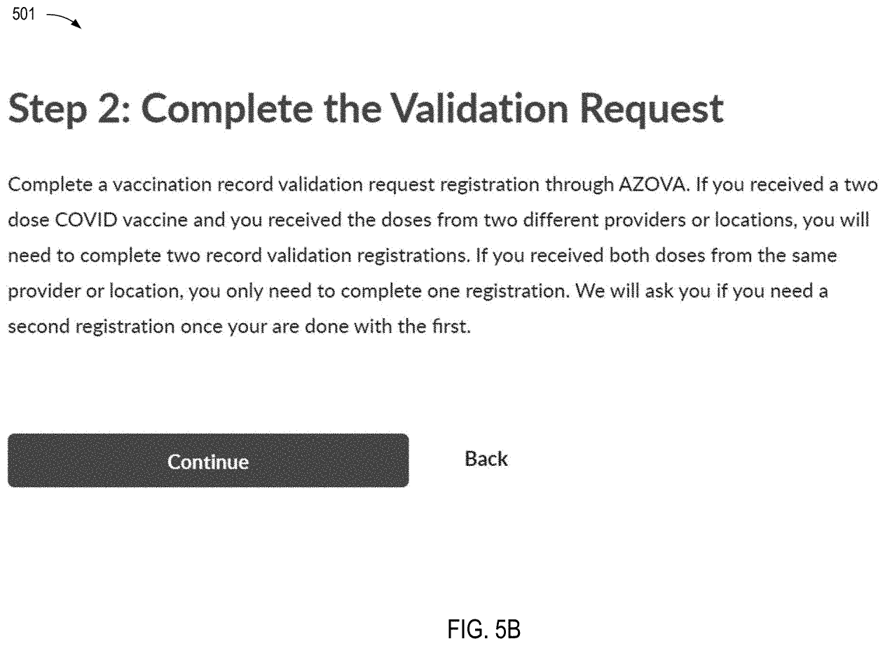

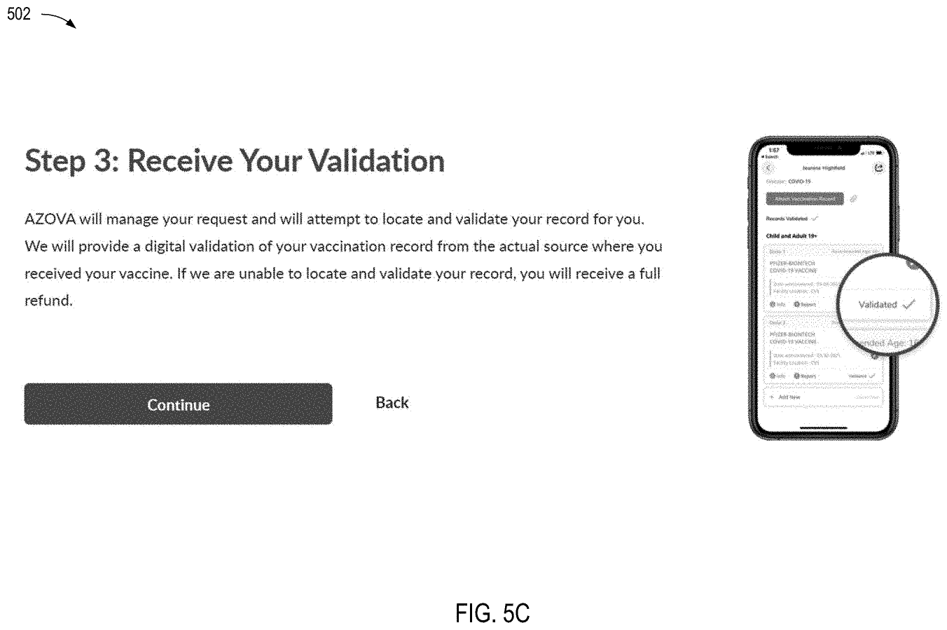

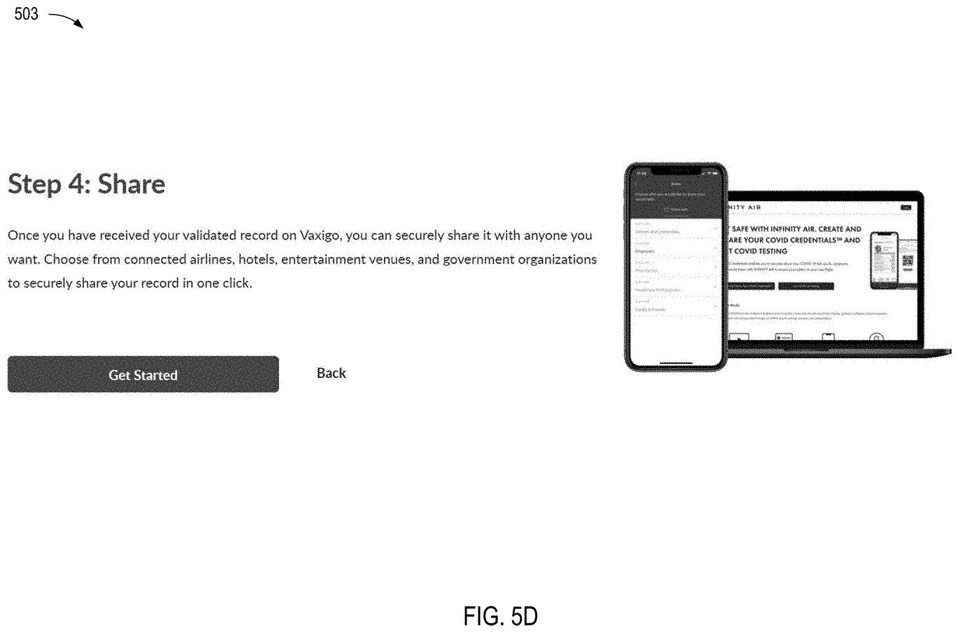

FIG. 5A illustrates an example GUI 500 for a computer-implemented process to locate a vaccine provider (or provider of another health credential).

FIG. 5B illustrates an example GUI 501 for a computer-implemented process for validating the user-identified or user-provided health credential.

FIG. 5C illustrates an example GUI 502 for a computer-implemented process for the user to receive and manage the sharing of the validated health credential.

FIG. 5D illustrates an example GUI 503 for a computer-implemented process for the user to share the user's personal information, the existence of one or more validated health credentials or a set of health credentials, and/or details of one or more validated health credentials or the details of a set of health credentials.

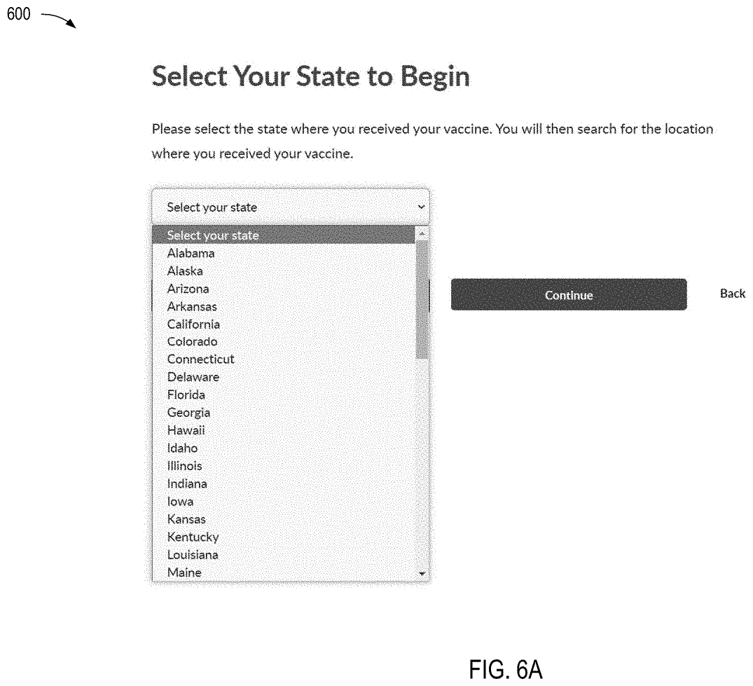

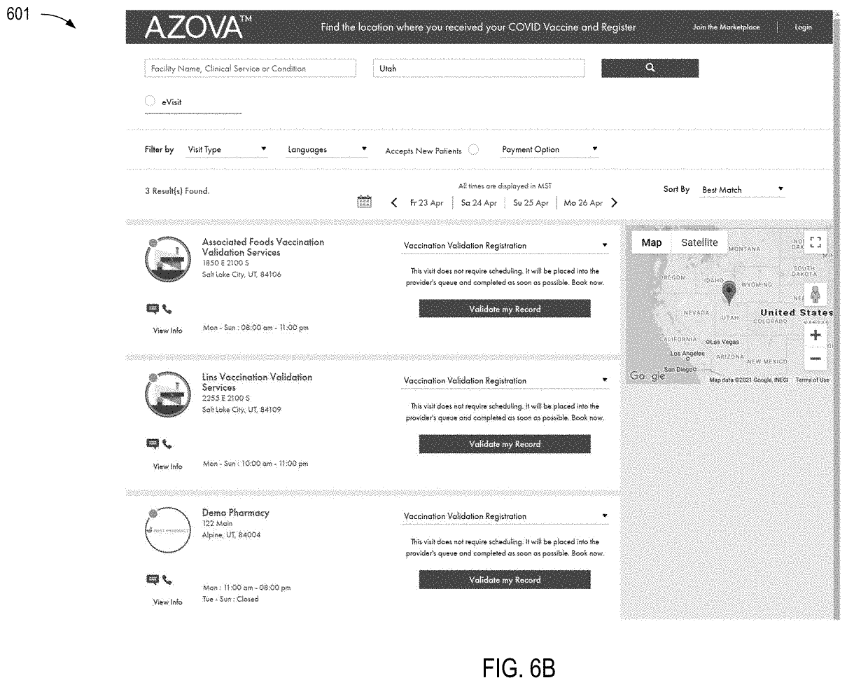

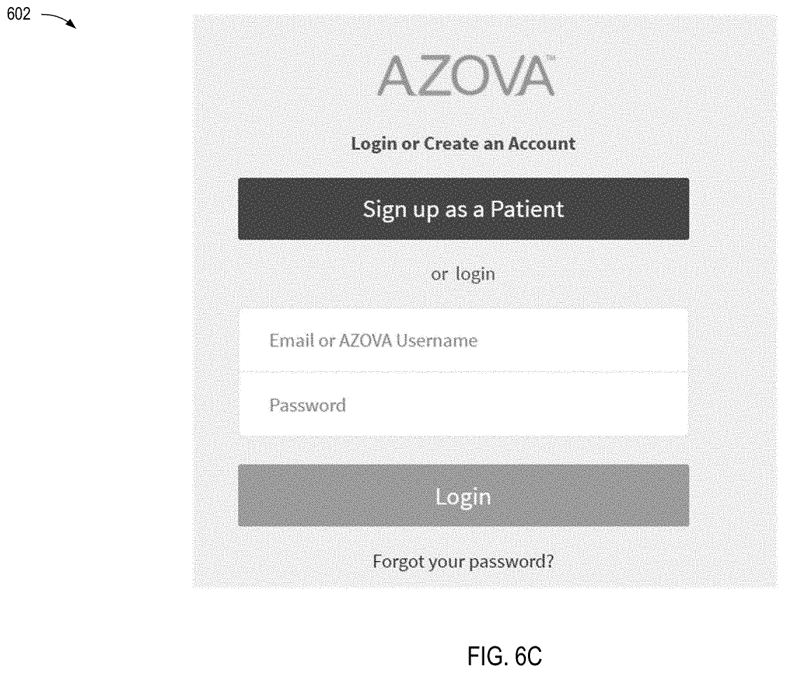

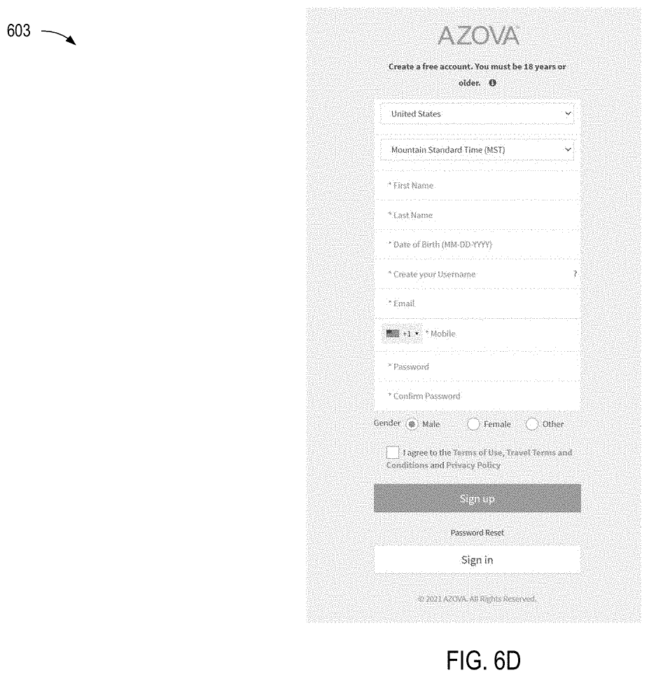

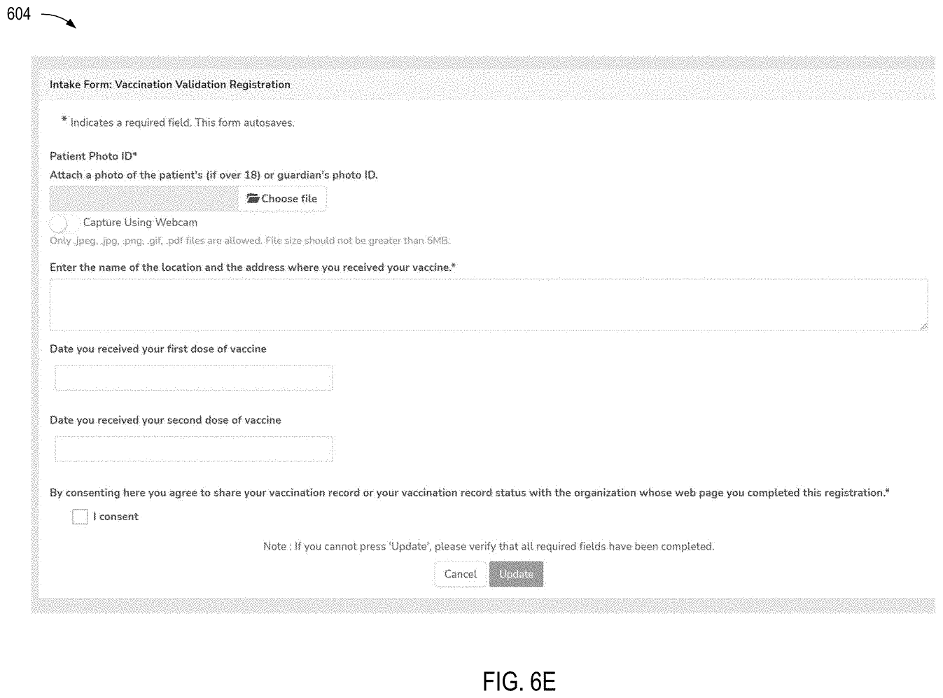

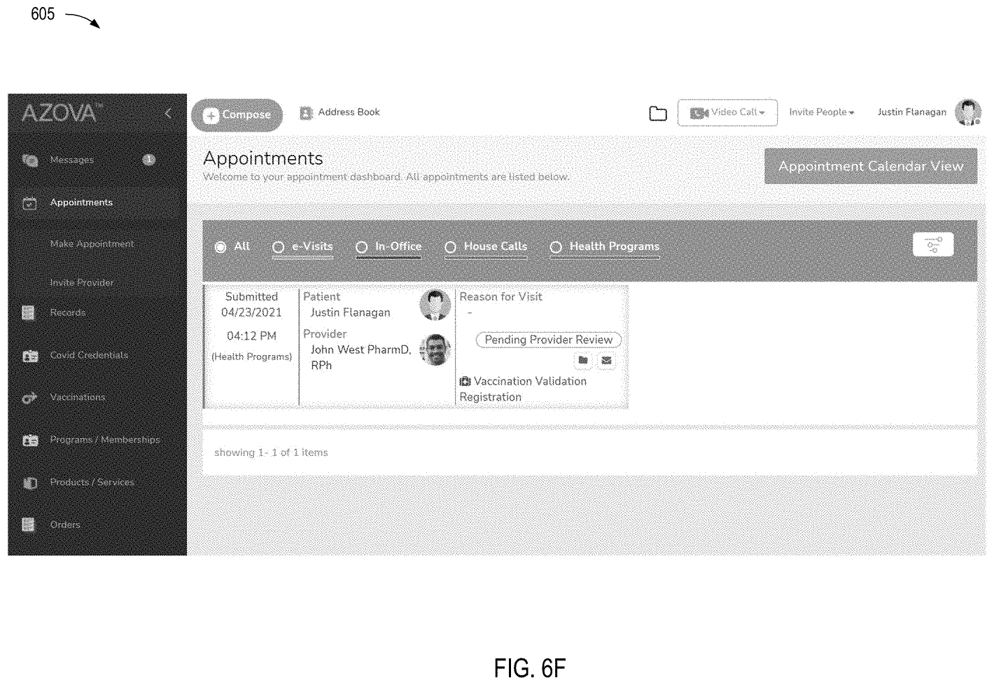

FIG. 6A illustrates an example GUI 600 for selecting a state within which a user asserts to have received a particular health credential or in which the user asserts a particular health credential can be validated or otherwise confirmed.

FIG. 6B illustrates an example GUI 601 of potential locations and map data of the locations from which the user can select as being able to confirm a particular health credential. For example, the user may identify the facility where the user received a Covid-19 vaccine.

FIG. 6C illustrates an example GUI 602 for logging in and/or creating an account, according to one embodiment.

FIG. 6D illustrates an example GUI 603 for providing the personal details to create an account, according to one embodiment.

FIG. 6E illustrates an example intake form 604 for a user to provide details of a vaccination for the system to then validate the vaccine to generate a validated vaccination health credential, according to one embodiment.

FIG. 6F illustrates a graphical user interface 605 of a pending validation as an appointment to be completed by a particular validating entity within the user account.

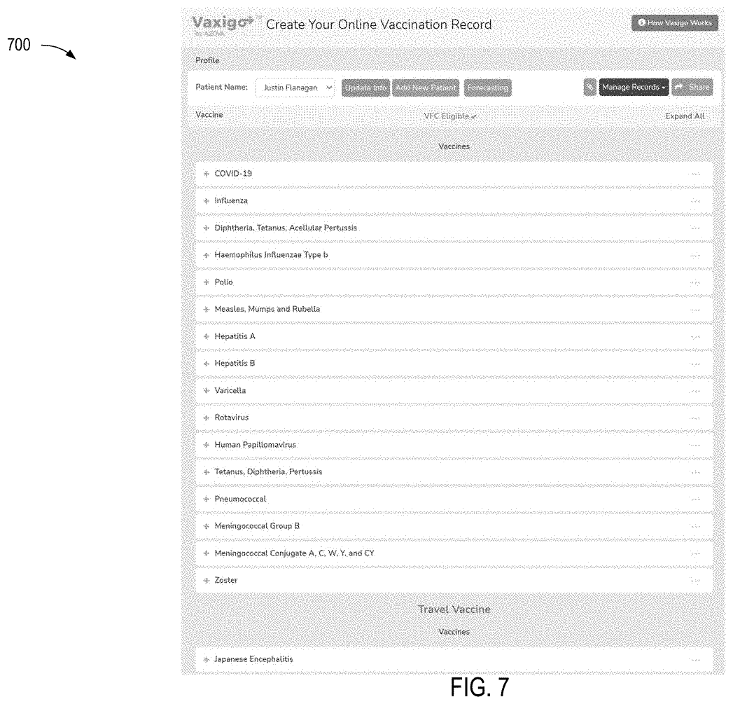

FIG. 7 illustrates an example GUI 606 for user-management and creation of vaccine records that can be validated and shared, according to one embodiment.

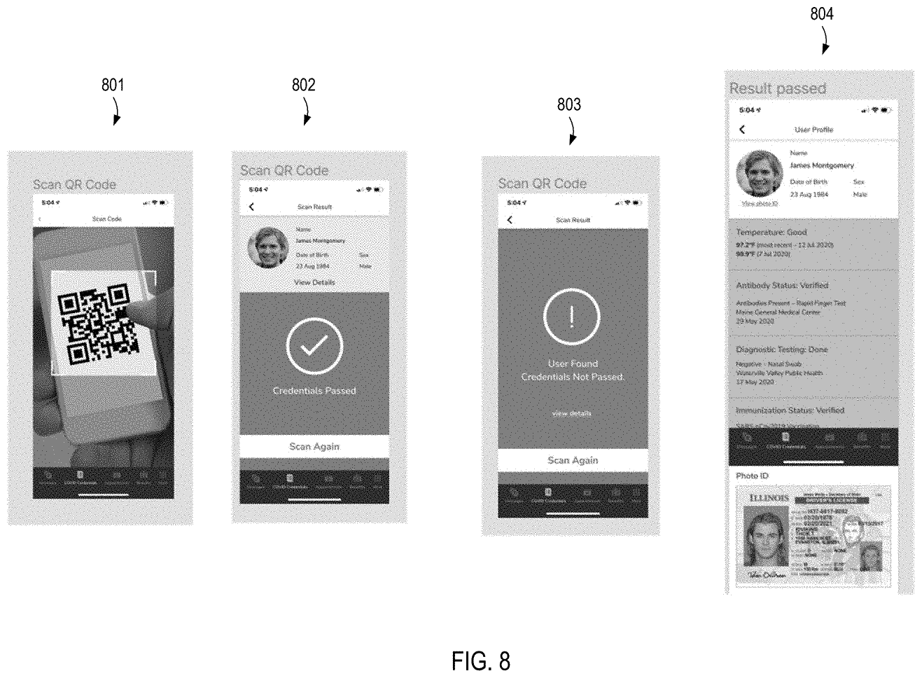

FIG. 8 illustrates series of graphical user interfaces, including a QR code interface 801 that can be used by the user to share personal health information, the details of one or more health credentials, and/or just the existence of one or more health credentials. The graphical user interface may further include a confirmation screen 802 on the user's device and/or the recipient's device indicates that the health credential satisfies predefined requirements. The graphical user interface may further include a screen indicating a failure to satisfy the recipients predefined requirements for health credentials, at 803. For example, the user may have a first does of a Covid-19 vaccine, but the receipt requires both doses of a Covid-19 vaccine. As another example, the user may have received a negative Covid-19 antigen test, but the receipt requires a negative PCR Covid-19 test. The graphical user interface may further include a screen, at 804, in which additional details of the user's health credentials along with a photo for identification purposes. In some embodiments, some details of the user's personal information may be redacted.

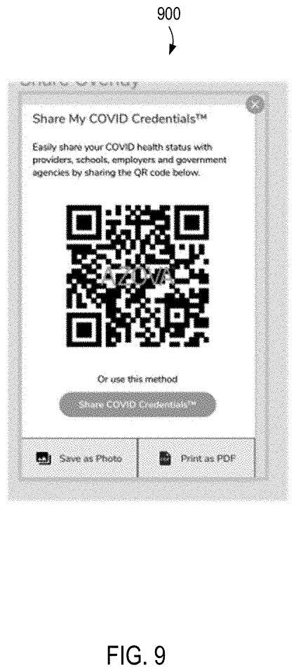

FIG. 9 illustrates an example GUI 900 for a user to share vaccination credentials (or any other health credential) as a photo, via an app, via email, as a printed PDF, or as QR code, according to one embodiment.

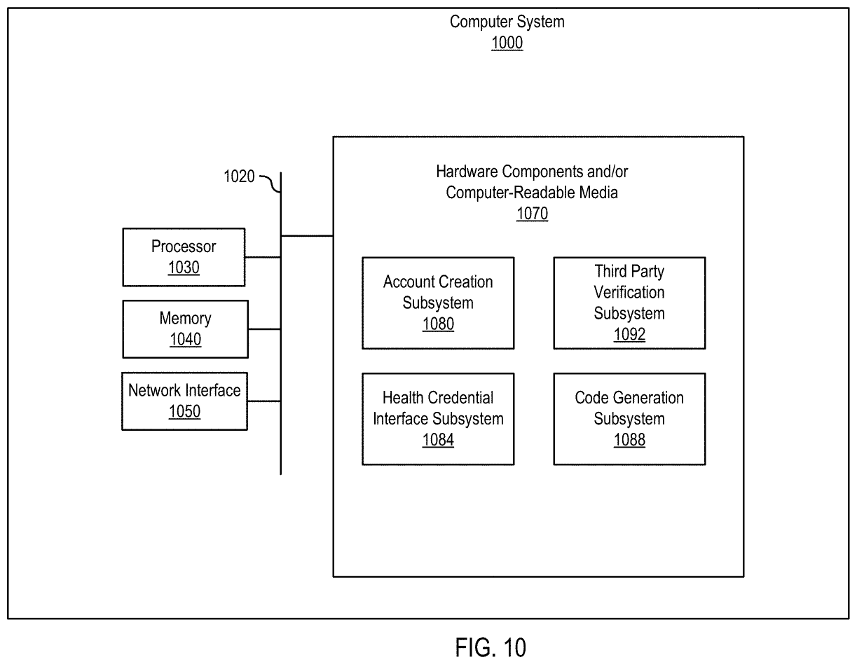

FIG. 10 illustrates an example of a computer system 1000 that includes a processor 1030, memory 1040, network interface 1050, and a computer-readable media 1070. The computer readable media 1070 may include modules or subsystems and be connected to, for example, the processor 1030 via a bus 1020. The computer readable media 1070 may include an account creation subsystem 1080, a third party verification subsystem 1092, a health credential interface subsystem 1084, and a code generation subsystem 1088. The account creation subsystem 1080 may be used to create a user account.

The health credential interface subsystem 1084 may be used to receive user-provided health credentials, such as: (0 a user-provided health credential identifying a vaccination status of the user with respect to a specific disease, (ii) information identifying a first entity that provided the vaccination status of the user (iii) a user-provided health credential identifying a test status of the user with respect to the specific disease, and (iv) information identifying a second entity that administered the test to the user for the specific disease.

The third-party verification subsystem 1092 may be used to contact the first entity to verify the vaccination status of the user and contact the second entity to verify the test status of the user. The code generation subsystem 1088 may be used to generate a single OR code that can be scanned by a verifying entity. The single OR code can provide the verifying entity with a combined verification of the vaccination status of the user and the test status of the user with respect to the specific disease. For example, the single code can be used to confirm a vaccination status and a negative test result with respect to a specific disease, such as COVID-19.

Each of the graphical user interfaces (GUIs) described herein may be implemented and/or generated by a modules or subsystems on personal computing devices, personal computers, and/or backend servers. Each module or subsystem may be named or referred to by the function that it facilitates and may be part of, for example, a hardware system and/or a computer-readable medium.

This disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element.

1. A test kit, comprising:
a packaging;
a saliva collection container within the packing, wherein the saliva collection container includes a unique identifier code; and
a lab order coupon code within the packaging that includes information to associate the unique identifier code of the saliva collection container with an online user account.

2. A system comprising:
a processor;
an account creation subsystem to generate a first graphical user interface for rendering on a user electronic device, the first graphical user interface to be utilized by a user to create a user account;
a health credential interface to generate a second graphical user interface to receive:
(i) a user-provided health credential identifying a vaccination status of the user with respect to a specific disease,
(ii) information identifying a first entity that provided the vaccination status of the user,
(iii) a user-provided health credential identifying a test status of the user with respect to the specific disease, and
(iv) information identifying a second entity that administered the test to the user for the specific disease;
a third-party verification subsystem to:
contact the first entity to verify the vaccination status of the user, and
contact the second entity to verify the test status of the user; and
a code generation subsystem to generate a single scannable verification code that can be scanned by a verifying entity, the single scannable verification code providing the verifying entity with a combined verification of:
(i) the vaccination status of the user, and
(ii) the test status of the user with respect to the specific disease,
wherein the single scannable verification code redacts at least one item of personal information about the user such that the redacted information is not visible to the verifying entity scanning the single scannable verification code.

3. A non-transitory computer-readable medium with instructions stored therein that, when executed by a processor of a computing system, cause the system to implement operations to:
facilitate the creation, by a user, of a user account;
receive user-provided details associated with a health credential;
add the health credential to the user account as a user-provided health credential;
contact a third party and submit a request o validate the user-provided health credential;
receive, from the third party, validation of the user-provided health credential;
replace the user-provided health credential with a validated health credential based on the third-party validation;
receive a request by the user to share the existence of the validated health credential with an entity; and
indicate to the entity that the user has the validated health credential.

4. The non-transitory computer-readable medium of claim 3, wherein the operation to contact a third-party to request the validation comprises querying a third-party database to validate the user-provided health credential.

5. The non-transitory computer-readable medium of claim 3, wherein the operation to contact a third-party to request the validation comprises requesting manual verification of a health credential by an employee of the third party.

6. A non-transitory computer-readable medium with instructions stored therein that, when executed by a processor of a computing device, cause the computing device to:
generate a graphical user interface accessible by a user to facilitate the creation of a user account by the user that includes personal identifying information;
receive, from the user, user-provided details associated with a profile credential, wherein the profile credential comprises non-personal identifying information;
add the profile credential to the user account as a user-provided profile credential;
request third-party validation from a third party of the user-provided profile credential;
receive, from the third party, validation of the user-provided profile credential; and
identify the user-provided profile credential within the user account as a validated profile credential based on the third-party validation.

7. The non-transitory computer-readable medium of claim 6, wherein the profile credential comprises a health credential.

8. The non-transitory computer-readable medium of claim 7, wherein the instructions are further configured to cause the computing device to:
receive a request by the user to share the existence of the validated health credential with an entity.

9. The non-transitory computer-readable medium of claim 8, wherein the instructions are further configured to cause the computing device to:
indicate to the entity that the user has the validated health credential.

10. The non-transitory computer-readable medium of claim 9, wherein the instructions are configured to cause the computing device to indicate to the entity that the user has the validated health credential by generating a OR code.

11. The non-transitory computer-readable medium of claim 10, wherein the instructions are configured to cause the computing device to generate the QR code together with a photo of the user.

12. The non-transitory computer-readable medium of claim 10, wherein the instructions are configured to cause the computing device to generate the QR code together with at least some of the personal identifying information.

13. The non-transitory computer-readable medium of claim 10, wherein the instructions are configured to cause the computing device to generate the OR code devoid of any personal identifying information.

14. The non-transitory computer-readable medium of claim 6, wherein the instructions are configured to cause the computing device to request third-party validation by querying a third-party database to validate the user-provided profile credential.

15. The non-transitory computer-readable medium of claim 6, wherein the instructions are configured to cause the computing device to request third-party validation by requesting manual verification of the profile credential by an employee of the third party.

16. The non-transitory computer-readable medium of claim 6, wherein the profile credential comprises a certification status.

17. The non-transitory computer-readable medium of claim 16, wherein the instructions are further configured to cause the computing device to;
receive a request by the user to share the existence of the validated certification status with an entity.

18. The non-transitory computer-readable medium of claim 17, wherein the instructions are further configured to cause the computing device to;
indicate to the entity that the user has the validated certification status.

19. The non-transitory computer-readable medium of claim 18, wherein the instructions are configured to cause the computing device to indicate to the entity that the user has the validated certification status by generating a QR code.

20. The non-transitory computer-readable medium of claim 19, wherein the instructions are configured to cause the computing device to generate the QR code together with a photo of the user.

21. The non-transitory computer-readable medium of claim 19, wherein the instructions are configured to cause the computing device to generate the OR code together with at least some of the personal identifying information.

22. The non-transitory computer-readable medium of claim 19, wherein the instructions are configured to cause the computing device to generate the OR code devoid of any personal identifying information.

23. The non-transitory computer-readable medium of claim 6, wherein the profile credential comprises one or more of a test score, a vaccination status, a test result, a certification status, and an approval status.
